(19) United States
(12) Patent Application Publication
(10) Pub. No.: US 2015/0374796 A1
(43) Pub. Date: Dec. 31, 2015

(54) USE OF HAEMOGLOBIN OF ANNELIDS FOR TREATING CANCER

(71) Applicant: HEMARINA, Morlaix (FR)

(72) Inventor: Franck ZAL, Ploujeanmorlaix (FR)

(21) Appl. No.: 14/768,303

(22) PCT Filed: Feb. 14, 2014
(86) PCT No.: PCT/FR2014/050300

§ 371 Date: Aug. 17, 2015

(30) Foreign Application Priority Data
Feb. 15, 2013 (FR) 13 51313

(51) Int. Cl. A61K 38/42; A61K 45/06

(52) U.S. Cl. CPC A61K 38/42; A61K 45/06

(57) ABSTRACT

A method for treating cancer using at least one globin, a globin protomer or an extracellular haemoglobin of annelids.

The present invention relates to the use of at least one extracellular annelid hemoglobin, globin, or globin protomer for treating cancer, in particular for reducing hypoxia in cancer cells, and thereby increasing the effectiveness of radiotherapy and chemotherapy treatments.

Cancer is a disease linked to the abnormal and uncontrollable proliferation of cells referred to as malignant.

Cancers encompass tumors referred to as solid tumors, i.e. in which the cells have multiplied to form a mass, and lymphomas and leukemias in which the cancer cells are circulating in the blood, i.e. do not therefore constitute a solid mass.

A reduction in the oxygen partial pressure (tissue hypoxia) has been demonstrated in almost all solid tumors in humans (Raleigh J, Dewhirst M, Thrall D. Measuring tumor hypoxia. Semin Radiat Oncol 1996; 46: 229-37). As early as the 1950s, tumoral architecture was described as a central hyaline necrosis surrounded by viable tissues, at a distance of approximately 100 μm from the capillaries (Thomlinson RH, Gray LH. The histological structure of some human lung cancers and possible implications for radiotherapy. Br J Cancer 1955; 9: 539.). Tumors have large regions with low oxygen partial pressure, in which the cells are under hypoxic conditions.

The heterodimeric transcription factor HIF-1 (hypoxia-inducible factor-1) activates a wide variety of signaling pathways enabling the cancer cell to acquire an adapted response to hypoxic stress. HIF-1 activates a series of more than 50 genes coding for protein factors involved, in particular, in neoangiongenesis, promoting tumor progression and metastatic dissemination, glucose metabolism (Glut-1), cell survival and chemoresistance (MDR) (Lauzier MC, Michaud MD, Déry MA, Richard DE. HIF-1 activation during tumor progression: implications and consequences. Bull Cancer 2006; 93: 349-56). Detection of the HIF-1 protein in tumors has been correlated with a reduction in survival rate and a reduction in sensitivity to chemotherapy. Indeed, cells deficient in the HIF-1α protein demonstrate increased sensitivity to numerous chemotherapy agents such as carboplatin or etoposide (Unruh A, Ressel A, Mohamed HG, Johnson RS, Nadrowitz R, Richter E, et al. The hypoxia-inducible factor-1 alpha is a negative factor for tumor therapy. Oncogene 2003; 22: 3213-20). Moreover, it has also been demonstrated that tumoral hypoxia stops the cell cycle in the G1/S phase, and enhances resistance to apoptosis by inactivating the tumor-suppressor gene p53. It has thus been demonstrated in a murine fibrosarcoma model that hypoxic cells are 2 to 6 times more chemoresistant than normoxic cells (Teicher BA, Holden SA, Al-Achi A, et al. Classification of antineoplastic treatments by their differential toxicity toward putative oxygenated and hypoxic tumor subpopulations in vivo in the FSaIIC murine fibrosarcoma. Cancer Res 1990; 50: 3339-44).

Hypoxia is also a major factor in radioresistance. Alterations to the nucleoside bases (single-strand and double-strand DNA breaks) may be produced “directly” by imparting energy to the DNA strands, or, more often, indirectly by the production of free radicals from the radiolysis of water. Oxygen is involved in this radical cascade by enabling the creation of longer-lasting free radicals.

Hypoxia could be considered to be an important target for therapy, given its high tumoral specificity (“L'hypoxie tumorale peut-elle devenir un avantage pour la chimiothérapie?” [Could tumoral hypoxia be advantageous to chemotherapy?], Tredan et al, Bulletin du Cancer, 2008, vol. 95, no.5, pp. 528-534).

New therapeutics are therefore being developed to target cells under hypoxic conditions. Numerous agents which may inhibit the expression or activity of HIF-1 are also in the preclinical or clinical phase.

In order to have effective therapies, it is relevant to identify therapies which inhibit hypoxia. Indeed, by inhibiting or reducing this mechanism, cancer cells may become sensitive to radiotherapy and chemotherapy treatments again.

There is therefore a need to reduce hypoxia in cancer cells, in cases of solid tumors.

The inventors have now discovered that, surprisingly, extracellular annelid hemoglobin enables hypoxia in cancer cells to be reduced, as is demonstrated in the example.

The present invention thus relates to the use of at least one molecule chosen from an extracellular annelid hemoglobin, globin, or globin protomer, for treating cancers, preferably solid tumors. Preferably, the cancers are treated by reducing hypoxia in the cancer cells; this makes them more sensitive to radiotherapy and chemotherapy treatments. The present invention also relates to at least one extracellular annelid hemoglobin globin, or globin protomer, for the use thereof for reducing hypoxia in cancer cells.

The present invention thus relates to a product consisting of an anti-cancer agent and an extracellular annelid hemoglobin, globin, or globin protomer, as a combined preparation for simultaneous, separate or sequential use for treating cancer.

Preferably, the anti-cancer agent is chosen from fludarabine, gemcitabine, capecitabine, methotrexate, taxol, taxotere, mercaptopurine, thioguanine, hydroxyurea, cytarabine, cyclophosphamide, ifosfamide, nitrosoureas such as carmustine and lomustine, platinum complexes such as cisplatin, carboplatin and oxaliplatin, mitomycin, dacarbazine, procarbizine, etoposide, teniposide, campathecines, bleomycin, doxorubicin, idarubicin, daunorubicin, dactinomycin, plicamycin, mitoxantrone, L-asparaginase, epimbicm, 5-fluorouracil, taxanes such as docetaxel and paclitaxel, leucovorin, levamisole, irinotecan (CPT-11), SN-38, estramustine, mustine hydrochloride, BCNU, vinblastine, vincristine and vinorelbine, anti-EGF receptor or anti-VEGF receptor monoclonal antibodies such as bevacizumab, cetuximab and panitumumab, imatimb mesylate, hexamethyhnelamine, topotecan, genistein, erbstatin, lavendustin and also bortezomib (or PS341, sold by Millenium Pharmaceuticals under the name Velcade).

The extracellular annelid hemoglobin is present in the three classes of annelids: the polychaetes, the oligochaetes and the hirudinea. Reference is made to extracellular hemoglobin because it is not naturally contained in a cell, and can therefore circulate freely in the bloodstream without chemical modification to stabilize it or make it functional.

The extracellular annelid hemoglobin is a giant biopolymer with a molecular weight of between 2000 and 4000 kDa, made up of approximately 200 polypeptide chains of between 4 and 12 different types which are generally grouped into two categories.

The first category, with 144 to 192 components, groups together the “functional” polypeptide chains which bear an active site of heme type, and are capable of reversibly binding oxygen; these are chains of globin type, the weights of which are between 15 and 18 kDa and which are very similar to the α- and β-type chains of vertebrates.

The second category, with 36 to 42 components, groups together the “structural” or “linker” polypeptide chains which have few or no active sites but enable the assembly of the subunits called one-twelfth subunits or protomers.

Each hemoglobin molecule consists of two superposed hexagons which have been named hexagonal bilayer, and each hexagon is itself formed by the assembly of six subunits (dodecamer or protomer) in the form of a drop of water. The native molecule is formed from twelve of these subunits (dodecamer or protomer). Each subunit has a molecular weight of around 250 kDa, and constitutes the functional unit of the native molecule.

Preferably, the extracellular annelid hemoglobin is chosen from the extracellular hemoglobins of polychaete annelids, and the extracellular hemoglobins of oligochaete annelids. Preferably, the extracellular annelid hemoglobin is chosen from the extracellular hemoglobins of the family Lumbricidae, the extracellular hemoglobins of the family Arenicolidae and the extracellular hemoglobins of the family Nereididae. Even more preferably, the extracellular annelid hemoglobin is chosen from the extracellular hemoglobin of Lumbricus terrestris, the extracellular hemoglobin of Arenicola sp and the extracellular hemoglobin of Nereis sp, more preferably the extracellular hemoglobin of Arenicola marina or of Nereis virens.

According to the invention, the globin protomer of the extracellular annelid hemoglobin constitutes the functional unit of native hemoglobin, as indicated above.

Finally, the globin chain of the extracellular annelid hemoglobin can in particular be chosen from the Ax and/or Bx type globin chains of extracellular annelid hemoglobin.

The extracellular annelid hemoglobin, globin protomers thereof and/or globins thereof do not require a cofactor in order to function, contrary to mammalian hemoglobin, in particular human hemoglobin. Finally, since the extracellular annelid hemoglobin, globin protomers thereof and/or globins thereof do not possess blood typing, they enable any problem of immunological reaction to be avoided.

The extracellular annelid hemoglobin, globin protomers thereof and/or globins thereof may be native or recombinant.

According to the invention, the extracellular annelid hemoglobin, globin or globin protomer is preferably present in a composition comprising a buffer solution.

Said buffer solution creates an appropriate saline environment for the hemoglobin, protomers thereof and globins thereof, and thus enables the quaternary structure and therefore the functionality of this molecule to be maintained. By virtue of the buffer solution, the hemoglobin, protomers thereof and globins thereof are capable of performing their oxygenation function.

The buffer solution according to the invention is an aqueous solution comprising salts, preferably chloride, sodium, calcium, magnesium and potassium ions, and gives the composition according to the invention a pH of between 6.5 and 7.6; its formulation is similar to that of a physiologically injectable liquid. Under these conditions, the extracellular annelid hemoglobin, globin protomers thereof and globins thereof remain functional.

In the present description, the pH is understood to be at ambient temperature (25° C.), unless otherwise indicated.

Preferably, the buffer solution is an aqueous solution comprising sodium chloride, calcium chloride, magnesium chloride, potassium chloride and also sodium gluconate and sodium acetate, and has a pH of between 6.5 and 7.6, preferably equal to 7.1 ± 0.5, preferably of approximately 7.35. More preferably, the buffer solution is an aqueous solution which comprises 90 mM of NaCI, 23 mM of Na-gluconate, 2.5 mM of CaCl₂, 27 mM of Na-acetate, 1.5 mM of MgCl₂, 5 mM of KCl, and has a pH of 7.1± 0.5, which can contain between 0 and 100 mM of antioxidant of ascorbic acid and/or reduced glutathione type.

Preferably, the composition is administered to the subject parenterally, preferably by injection or infusion.

Preferably, the composition comprising hemoglobin, protomers thereof or globins thereof, and the buffer solution, is administered as it is. Indeed, in this case, the hemoglobin, protomers thereof or globins thereof is (are) present in a composition comprising a buffer solution, which is preferably an aqueous solution comprising salts and which gives the composition a pH of between 6.5 and 7.6. Preferably, the composition contains only hemoglobin, protomers thereof or globins thereof and a buffer solution consisting of an aqueous solution comprising salts which gives the composition a pH of between 6.5 and 7.6. The forms of administration are therefore quite simple and effective.

Preferably, the cancers are chosen from carcinomas, sarcomas, melanomas, breast cancer, colon cancer, ovarian cancer, prostate cancer, uterine cancer, liver cancer, lung cancer and thyroid cancer.

The invention is described in more detail in the following examples. These examples are given purely by way of nonlimiting illustration.

The figure is illustrated by the following legend:

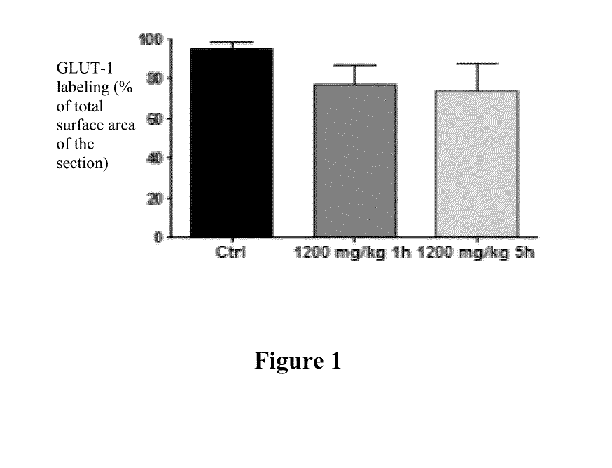

FIG. 1: Quantification of anti-GLUT-1 labeling on sections of tumor tissue in the control group (Ctrl), and 1 h and 5 h after i.v. injection of 1200 mg/kg M101. The positive signal observed on the paraffin sections was expressed as a fraction of the area of labeled tumor cells compared to the total surface area of the tumor. The values correspond to the mean of 3 samples (mean +/− SD).

### EXAMPLE

### Materials:

### Arenicola marina (M101):

Two batches at concentrations of 100 mg/ml and 178 mg/ml were used for this study. M101 is stored at −80° C. and thawed at 4° C. for the studies.

M101 was diluted to the concentration studied with the M101 stabilizing buffer: 4 mM KCl, 145 mM NaCl, 0.2 mM MgCl2, 10 mM HEPES, 0.1 M NaOH; pH 7.

### In Vivo Tumor Model, Treatment with M101 and Immunohistochemical Analyses of the Tumors:

HT29 human colonic adenocarcinoma cells (ATCC, HTB-38) were grown in DMEM supplemented with 20% FBS, 1% L-glutamine and 1% antibiotics (penicillin and streptomycin) at 37° C. in an atmosphere saturated with water, at 5% CO₂/95% air.

The HT29 cells, amplified in vitro (3 × 10⁶ cells), were injected subcutaneously in the region of the right flank of Nude mice 6-8 weeks old, which had been irradiated beforehand at a dose of 5 Gy. When the diameter of the tumors reached ˜5 mm, the mice were injected intravenously with M101 at 3 different concentrations (60, 600 and 1200 mg/kg). The ability of M101 to reduce hypoxia in the tumors was monitored over time, by quantification of the expression of GLUT-1 using immunohistochemistry (anti-GLUT-1 antibody and detection by streptavidin-biotin). For this purpose, the mice were euthanized at different times after treatment with M101 and samples were taken from the HT29 tumors at 5 min, 15 min, then at 1, 2, 3, 4, 5, 6 and 24 h. GLUT-1 was evaluated as an intrinsic marker of hypoxia in the tissues (Gbadamosi JK, Hunter AC, Moghimi SM (2002) PEGylation of microspheres generates a heterogeneous population of particles with differential surface characteristics and biological performance. FEBS Lett 532:338-344; Pionetti JM, Pouyet J (1980) Molecular architecture of annelid erythrocruorins. Extracellular hemoglobin of Arenicola marina (Polychaeta). Eur J Biochem 105:131-138).

### Results

### Kinetics of Oxygenation of Tumor Tissue

The oxygenating capacity of M101 was determined in vivo by evaluating the reduction of the degree of hypoxia in the subcutaneous HT29 tumors by immunhistological quantification of the GLUT-1 marker (table 1 below).

TABLE 1

| | Injected dose (mg/kg) | | |
|---|---|---|---|
| | 60 | 600 | 1200 |
| Control | +++ | +++ | +++ |
| 5 min | +++ | ++/− | +/− |
| 15 min | +++ | ++/− | +/− |
| 1 h | +++ | +/− | + |
| 2 h | +++ | + | + |
| 3 h | +++ | +/− | + |
| 4 h | +++ | +/− | + |
| 5 h | +++ | +/− | +/− |
| 6 h | ++/− | + | + |
| 24 h | +++ | +/− | +/− |

The degree of tissue hypoxia is evaluated by quantification of GLUT-1 labeling over time, following intravenous injection of M101.
The degree of tissue hypoxia is determined according to the following scale:
“+++” = very intense Glut-1 labeling, very high tissue hypoxia
“++/−” = intense Glut-1 labeling, high tissue hypoxia
“+”, “+/−” = intermediate Glut-1 labeling, reduced tissue hypoxia
“+/−” = low Glut-1 labeling, low tissue hypoxia

Following digitization of the histological slides, dedicated software enabled the degree of hypoxia for each condition to be measured. Examination of tumor tissue samples after treatment (injection of M101 at 600 mg/kg and 1200 mg/kg) demonstrated that the hypoxic regions had been reduced compared to the controls, and replaced by fibrous tissue which tends to dissociate and infiltrate the tumor. Intravenous administration of M101 at a dose of 1200 mg/kg reduces the degree of tumor hypoxia by 20% on average after 1 hour (rising as high as 40%) and 23% after 5 hours (see FIG. 1).

These data suggest that M101 is capable of diffusing into the tumor tissue and of reducing the intensity of GLUT-1 staining, thereby demonstrating its effect on tumor oxygenation.

1-10. (canceled)

11. A method for treating cancer in a patient in need thereof, comprising administering a molecule chosen from an extracellular annelid hemoglobin, globin, or globin protomer to said patient.

12. The method as claimed in claim 11, wherein the molecule reduces hypoxia in cancer cells.

13. A method for treating cancer in a patient in need thereof, comprising administering simultaneously, separately or sequentially a product consisting of an anti-cancer agent and an extracellular annelid hemoglobin, globin, or globin protomer.

14. The method as claimed in claim 11, wherein the extracellular annelid hemoglobin is chosen from the extracellular hemoglobins of polychaete annelids.

15. The method as claimed in claim 11, wherein the extracellular annelid hemoglobin is chosen from the extracellular hemoglobins of the family Lumbricidae, the extracellular hemoglobins of the family Arenicolidae and the extracellular hemoglobins of the family Nereididae.

16. The method as claimed in claim 11, wherein the extracellular annelid hemoglobin is chosen from the extracellular hemoglobin of Lumbricus terrestris, the extracellular hemoglobin of Arenicola sp and the extracellular hemoglobin of Nereis sp.

17. The method as claimed in claim 11, wherein the extracellular annelid hemoglobin is chosen from the extracellular hemoglobin of Arenicola marina and the extracellular hemoglobin of Nereis virens.

18. The method as claimed in claim 11, wherein the cancers are solid tumors.

19. The method as claimed in claim 11, wherein the cancers are chosen from carcinomas, sarcomas, melanomas, breast cancer, colon cancer, ovarian cancer, prostate cancer, uterine cancer, liver cancer, lung cancer and thyroid cancer.

20. The method as claimed in claim 11, wherein the hemoglobin is present in a composition comprising a buffer solution, which is preferably an aqueous solution comprising salts and which gives the composition a pH of between 6.5 and 7.6.

21. The method as claimed in claim 13, wherein the extracellular annelid hemoglobin is chosen from the extracellular hemoglobins of polychaete annelids.

22. The method as claimed in claim 13, wherein the extracellular annelid hemoglobin is chosen from the extracellular hemoglobins of the family Lumbricidae, the extracellular hemoglobins of the family Arenicolidae and the extracellular hemoglobins of the family Nereididae.

23. The method as claimed in claim 13, wherein the extracellular annelid hemoglobin is chosen from the extracellular hemoglobin of Lumbricus terrestris, the extracellular hemoglobin of Arenicola sp and the extracellular hemoglobin of Nereis sp.

24. The method as claimed in claim 13, wherein the extracellular annelid hemoglobin is chosen from the extracellular hemoglobin of Arenicola marina and the extracellular hemoglobin of Nereis virens.

25. The method as claimed in claim 13, wherein the cancers are solid tumors.

26. The method as claimed in claim 13, wherein the cancers are chosen from carcinomas, sarcomas, melanomas, breast cancer, colon cancer, ovarian cancer, prostate cancer, uterine cancer, liver cancer, lung cancer and thyroid cancer.

27. The method as claimed in claim 13, wherein the hemoglobin is present in a composition comprising a buffer solution, which is preferably an aqueous solution comprising salts and which gives the composition a pH of between 6.5 and 7.6.
